(19) United States
(12) Patent Application Publication
(10) Pub. No.: US 2009/0048518 A1
(43) Pub. Date: Feb. 19, 2009

(54) DOPPLER MOTION SENSOR APPARATUS AND METHOD OF USING SAME

(75) Inventor: Dan Gur Furman, Gedera (IL)

Correspondence Address: BAKER & DANIELS LLP, 300 NORTH MERIDIAN STREET, SUITE 2700, INDIANAPOLIS, IN, 46204, US

(73) Assignee: Cardio Art Technologies Ltd., Yehuda (IL)

(21) Appl. No.: 12/206,885

(22) Filed: Sep. 9, 2008

Related U.S. Application Data
(63) Continuation-in-part of application No. 12/119,339, filed on May 12, 2008.
Application No. 12/119,339 is a continuation-in-part of application No. PCT/IL2006/001416, filed on Dec. 10, 2006.

(30) Foreign Application Priority Data
Aug. 30, 2007 (IL) 185609

(51) Int. Cl. A61B 8/06

(52) U.S. Cl. 600/454; 600/438

(57) ABSTRACT

An apparatus for, and method of, sensing characteristics of a vessel and a fluid conveyed therein.

### CROSS-REFERENCE TO RELATED APPLICATIONS

The present application claims priority to and is a continuation-in-part of U.S. patent application Ser. No. 12/119,339 titled “DOPPLER MOTION SENSOR APPARATUS AND METHOD OF USING SAME” filed May 12, 2008, which is a continuation-in-part of Israel Patent Application No. 185609 filed Aug. 30, 2007, titled “MULTI FUNCTION SENSSOR,” and International Patent Application No. PCT/IL2006/001416 filed Dec. 10, 2006, titled “IMPLANTABLE BIOSENSING DEVICE AND HEALTH MONITORING SYSTEM AND METHOD INCLUDING SAME,” which claims the benefit of U.S. Provisional Patent Application Ser. No. 60/748,218 filed Dec. 8, 2005, titled “WIRELESS INTEGRATED TRANSMITTER AND SENSOR,” the entire disclosure of each application being expressly incorporated by reference herein.

The present application is related to U.S. Utility patent application Ser. No. 12/119,315 titled “OPTICAL SENSOR APPARATUS AND METHOD OF USING SAME” (hereinafter, “the Optical Sensor Application”), filed May 12, 2008, U.S. Utility patent application Ser. No. 12/119,325 titled “INTEGRATED HEART MONITORING DEVICE AND METHOD OF USING SAME” (hereinafter, “the Integrated Heart Application”), filed May 12, 2008, U.S. Utility patent application Ser. No. 12/119,462 titled “METHOD AND SYSTEM FOR MONITORING A HEALTH CONDITION” (hereinafter, “the Health Condition Application”), filed May 12, 2008 and U.S. patent application Ser. No. 12/096,474 titled “IMPLANTABLE BIOSENSING DEVICE AND HEALTH MONITORING SYSTEM AND METHOD INCLUDING SAME” filed Jun. 6, 2008, the entire disclosure of each application being expressly incorporated by reference herein.

### FIELD OF THE APPLICATION

The present invention relates to sensing devices and, more specifically, to sensing devices for sensing the velocity of fluids.

### BACKGROUND

For medical reasons, in vivo parameters of a patient may need to be monitored over a period of time. Heart arrhythmias are changes in the normal sequence of electrical impulses that cause the heart to pump blood through the body. Continuous monitoring may be required to detect arrhythmias because abnormal heart impulse changes might only occur sporadically. With continuous monitoring, medical personnel can characterize cardiac conditions and establish a proper course of treatment.

One prior art device that measures heart rate is the “Reveal” monitor by Medtronic (Minneapolis, Minn., USA). This device comprises an implantable heart monitor used, for example, in determining if syncope (fainting) in a patient is related to a heart rhythm problem. The Reveal monitor continuously monitors the rate and rhythm of the heart for up to 14 months. After waking from a fainting episode, the patient places a recording device external to the skin over the implanted Reveal monitor and presses a button to transfer data from the monitor to the recording device. The recording device is provided to a physician who analyzes the information stored therein to determine whether abnormal heart rhythm has been recorded. The use of the recording device is neither automatic nor autonomic, and therefore requires either the patient to be conscious or another person's intervention to transfer the information from the monitor to the recording device.

Another known type of implantable monitoring device is a transponder-type device, in which a transponder is implanted in a patient and is subsequently accessed with a hand-held electromagnetic reader in a non-invasive manner. An example of the latter type of device is described in U.S. Pat. No. 5,833,603.

### SUMMARY

A sensing device for acquiring signals and computing measurements is disclosed herein. In one embodiment, the sensing device includes a sensor having one or more transducers for transmitting and receiving acoustic energy and converting the received acoustic energy into one or more signals. The sensor is positioned facing a side of a vessel. A computing device operates the sensor and processes the plurality of signals to obtain measurement values. The sensor and the computing device are enclosed in a housing.

A method for acquiring signals and computing measurements is also disclosed herein. One embodiment of the method comprises the steps of providing a sensing device as disclosed in the paragraph above, transmitting acoustic energy from the one or more transducers, receiving acoustic energy from the one or more transducers to obtain one or more signals, processing the one or more signals to obtain measurement values, and analyzing the measurement values to obtain a parameter value indicative of a characteristic of the fluid.

In another embodiment, a device for acoustically measuring a characteristic of at least one of a blood vessel and blood flowing through the blood vessel is provided. The device includes a housing having a first side and a second side, a sensor assembly, and a computing device. The sensor assembly is mounted to the housing and includes one or more transducers for transmitting acoustic energy through the first side of the housing, receiving acoustic energy though the first side of the housing, and converting the acoustic energy into signals. The computing device is configured to activate the one or more transducers and interpret the signals to determine the characteristic. The housing encloses the sensor and the computing device.

The features of this invention, and the manner of attaining them, will become more apparent and the invention itself will be better understood by reference to the following description of embodiments of the invention taken in conjunction with the accompanying drawings.

### BRIEF DESCRIPTION OF THE DRAWINGS

FIG. 1A is a schematic side view of an exemplary embodiment of a sensing device;

FIG. 1B is an outwardly-facing view of the sensing device of FIG. 1;

FIG. 1C is a perspective view of the sensing device of FIG. 1;

FIGS. 2 and 3 are schematic side views of the sensing device of FIG. 1 and a vessel;

FIG. 4 is a schematic top-side view of an exemplary embodiment of a Doppler sensor;

FIG. 5 is a conceptual vector representation of wave and fluid flow orientations;

FIGS. 6A-6D are schematic front, side, top, and perspective views, respectively, of a Doppler sensor according to another exemplary embodiment;

FIG. 7 is a schematic top view of another exemplary embodiment of a Doppler sensor;

FIG. 8 is a conceptual view of a system adapted to transmit and receive communication signals from the sensing device of FIG. 1;

FIG. 9 is a flow-chart of an exemplary method for measuring motion;

FIG. 10 is a schematic representation of a cardiac cycle;

FIG. 11 is a conceptual view of fluid flowing through a vessel;

FIG. 12 is a graph of a measurements taken during a cardiac cycle; and

FIG. 13 is a conceptual view of a Doppler sensor according to an exemplary embodiment.

Corresponding reference characters indicate corresponding parts throughout the several views. Although the drawings represent embodiments of the present invention, the drawings are not necessarily to scale and certain features may be exaggerated in order to better illustrate and explain the present invention. The exemplifications set out herein illustrate embodiments of the invention in several forms and such exemplification is not to be construed as limiting the scope of the invention in any manner.

### DETAILED DESCRIPTION

The embodiments discussed below are not intended to be exhaustive or limit the invention to the precise forms disclosed in the following detailed description. Rather, the embodiments are chosen and described so that others skilled in the art may utilize their teachings.

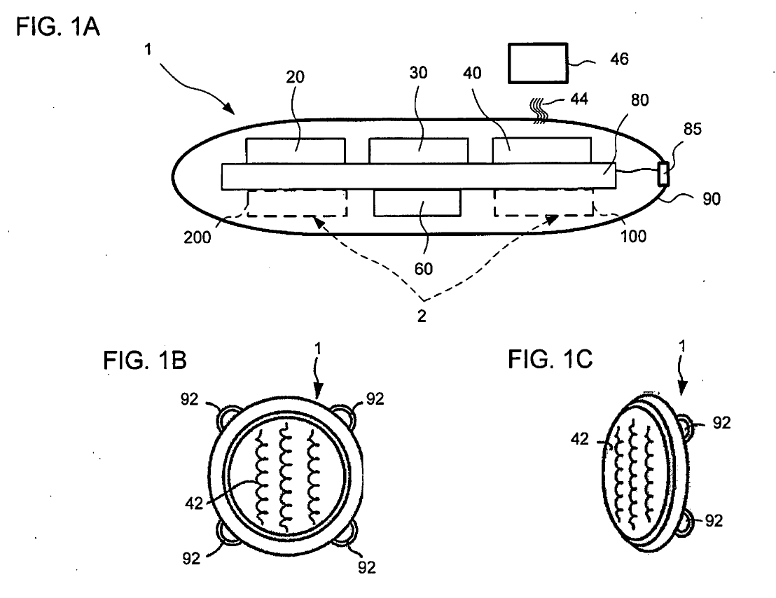

FIG. 1A illustrates a sensing device 1 according to one exemplary embodiment. Sensing device 1 generally includes a plurality of components including a Doppler sensor 60, a computing device 20, a communication device 30, and an energy storage device 40, each of the components mounted on a board 80 and being in electronic communication with computing device 20. The components are enclosed in a housing 90. In one embodiment, energy storage device 40 is adapted to receive electromagnetic energy waves 44 from an external energy source 46.

In one embodiment, sensing device 1 is adapted to determine a physiological condition of a patient. By “patient” it is meant a person or animal whose physiological condition is measured by sensing device 1. Although the invention disclosed herein is described in the medical context, the teachings disclosed herein are equally applicable in other contexts where compact data acquisition assemblies are desirable to perform measurements over time. For example, sensor assemblies may be desirable in submersed or difficult to reach applications, in dangerous environments, in applications having weight and size restrictions, in field research activities, and so on.

In one embodiment, sensing device 1 is implanted subcutaneously in the patient's body. It should be understood, however, that sensing device 1 may be implanted at different locations using various implantation techniques. For example, sensing device 1 may be implanted within the chest cavity beneath the rib cage. Housing 90 may be formed in the shape of a circular or oval disc, with dimensions roughly the same as two stacked quarter dollar coins. Of course, housing 90 may be configured in a variety of other shapes, depending upon the application. It may include four outwardly projecting loops 92, shown in FIGS. 1B and 1C, for receiving sutures in order to fix the assembly subcutaneously within the patient's body. More or fewer loops 92 may be provided depending upon the shape of housing 90. When so fixed, Doppler sensor 60 is positioned facing inwardly while an energy coupler, which is described with particularity below, faces outwardly.

In another embodiment of a sensing device 1, Doppler sensor 60 and other features of the sensing device 1 are integrated with an implanted cardiac device such as a pacemaker, a Cardiac Resynchronization Therapy (CRT) device, an implantable cardioverter defibrillator (ICD), etc. In one embodiment, integration may be achieved by combining the components of the sensing device and the cardiac device. If the cardiac device includes a computing device, for example, the algorithms that carry out the methods may be incorporated with the computing device of the cardiac device instead of adding a second computing device. In a similar manner, energy storage and communication devices may be combined to avoid duplication. In one embodiment, some components of the sensing device are included within the housing and some components are included with the cardiac device. The cardiac device and the components in the housing are operably connected.

In another embodiment, sensing device 1 is positioned externally to the patient's body. A support member is provided to support sensing device 1 externally to the body. The support member may be permanently or temporarily coupled to sensing device 1. In one embodiment, the support member comprises an adhesive layer for adhesively coupling the support member to the patient's body. In another embodiment, the support member comprises a belt, which may be elastic, for holding sensing device 1 against the patient's body.

Sensing device 1 may be implanted subcutaneously or positioned on the patient with the aid of an external mapping system such as an ultrasound machine. Proper placement ensures that a vessel of interest is located within the sensing range of sensing device 1. Where the vessel of interest is the aorta, sensing device 1 may be positioned on the chest or back of the patient in a location that reduces interference by the ribs of the measurements acquired in the manner described herein.

1. Doppler Sensor

A Doppler sensor comprises one or more transducers for insonating an object and receiving reflected ultrasonic waves. The velocity of a fluid of interest may be determined by directing an insonifying wave of ultrasonic energy towards the fluid at a known angle, measuring the frequency shift of the reflected ultrasound energy, and then calculating the velocity of the fluid. The Doppler frequency shift is proportional to the component of the velocity vector that is parallel to the insonifying wave. The velocity v of the fluid is determined by the following equation:

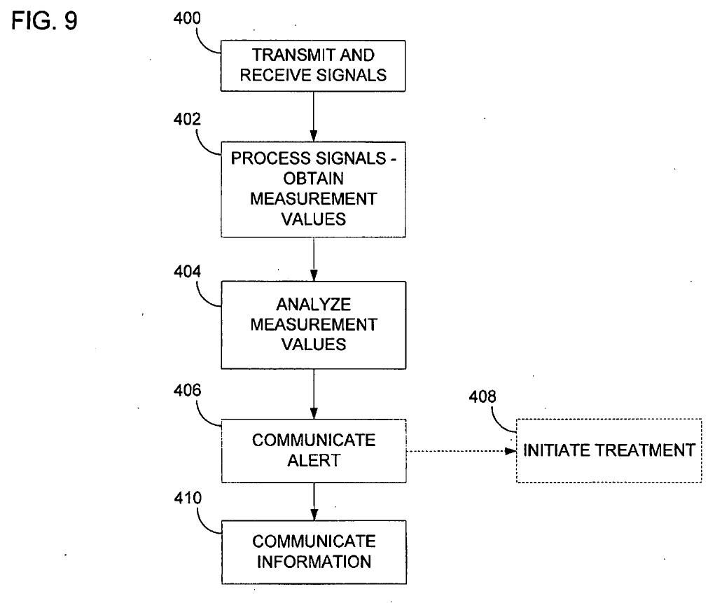

v=f_{d}·c/(2·f·cos θ)

where c is the velocity of sound in blood, f is the frequency of the insonifying wave, θ is the angle between the wave and the velocity vector, and f_{d} is Doppler frequency shift. Embodiments of a method to calculate blood pressure based on velocity measurements are described in full detail below with reference to FIGS. 9-12.

A transducer is a device which converts acoustic energy into electrical signals and vice-versa. Frequency shifts may be calculated by a variety of methods depending on the method of operation of the transducer(s). In one method of operation, the Doppler sensor may be a continuous wave sensor. A continuous wave Doppler sensor includes a transducer for transmitting ultrasonic waves and a transducer for receiving ultrasonic waves. The frequency shift in this method is measured directly by comparing the two waves. In another method, a pulsed wave Doppler sensor may be used. A pulsed wave Doppler sensor has a single transducer for transmitting and for receiving ultrasonic waves. After transmitting a wave, the Doppler sensor switches from a transmitting to a receiving mode of operation. The frequency shift is measured by comparing phase shifts between subsequently received waves. A plurality of waves transmitted and received in sequence are necessary to calculate the phase shifts. Well known algorithms, such as the Kasai or the cross-correlation algorithms, may be used to obtain the phase shift between the received and transmitted pulses.

Transducers may comprise coils, piezo-electric materials, and other suitable transducers. Transducers may be focused so as to transmit a narrow wave, or beam, of acoustic energy. Transducers may also transmit broad, or unfocused, waves of acoustic energy. Two or more transducers may be combined in a linear array to transmit an acoustic wave capable of insonating a large area with a desirable amount of energy. By large it is meant an area larger than what may be insonated with a single transducer. Linear arrays may be connected such that they may be driven as if they comprised a single transducer. Linear arrays may also be connected such that each transducer segment operates as an independent transducer.

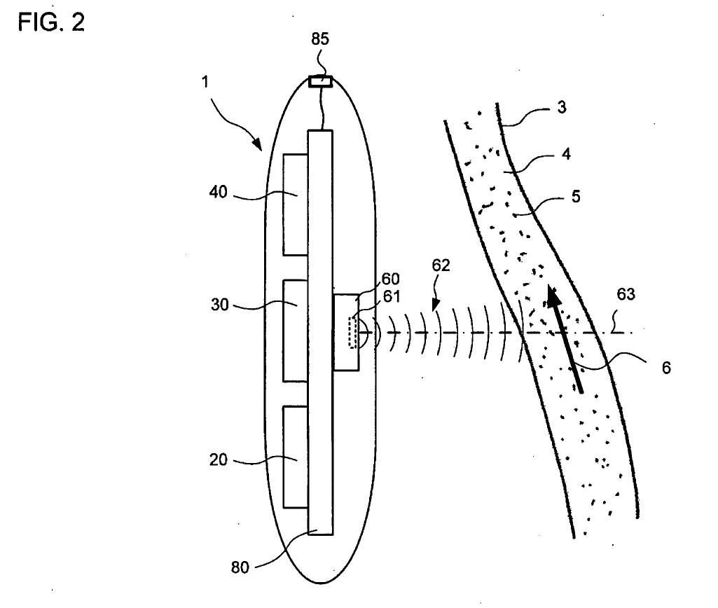

FIG. 2 illustrates the relationship between a vessel 3 conveying blood 4 having haemoglobin in red blood cells 5 and Doppler sensor 60. Doppler sensor 60 has transducer 61 positioned facing towards fluid 4 conveyed by vessel 3. Wave 62 transmitted by transducer 61 is shown propagating along a direction indicated by centerline 63 which is perpendicular to the surface of transducer 61. Arrow 6 indicates the direction of fluid 4 flow in vessel 3. While Doppler sensor 60 is described herein to describe its function in sensing assembly 1, other Doppler sensors described herein perform the same function and, in general, references to Doppler sensor 60 in this and related patent applications are equally applicable to other Doppler sensors described herein.

In one embodiment, a driver device, e.g., a pulse generator, provides an output corresponding to a desired frequency. The output may be amplified by an amplifier, such as a transistor, integrated with computing device 20 or provided externally of computing device 20. The output may comprise a wave form. Computing device 20 may provide the frequency generation function. In an alternative embodiment, a voltage is provided by the driver device to the transducer corresponding to a desired ultrasonic frequency and the transducer converts the electrical energy into acoustic energy in the form of an ultrasonic wave.

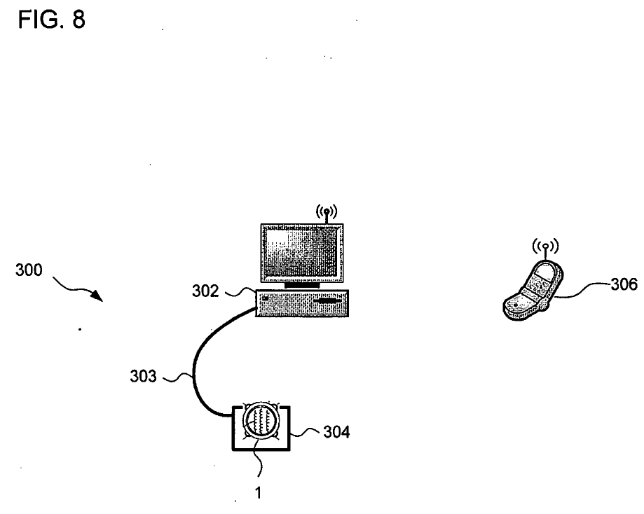

In one embodiment, sensing device 1 has a communication port for connecting to, and exchanging information with, other devices. Connector 85 is shown. The operation of connector 85, which is connected to other components of sensing device 1, is described in more detail further below with reference to FIG. 8.

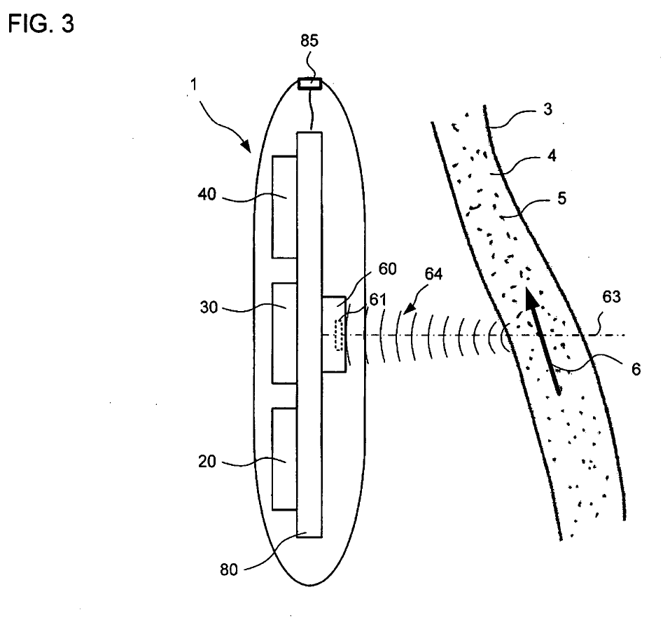

FIG. 3 illustrates reflected ultrasonic wave 64. Wave 64 is shown propagating along a direction indicated by centerline 63. Wave 64 propagates in a direction opposite that of wave 62. Wave 64 also has a frequency that is different from the frequency of wave 62. The difference is determined by the selection of transducers. In one embodiment, wave 62 is a continuous wave and wave 64 is reflected contemporaneously with wave 62. In another embodiment, wave 62 is a pulsed wave transmitted by transducer A before reflected wave 64 reaches transducer A. Computing device 20 may direct transducer A to transmit wave 62 and measure the time required for wave 64 to reach transmitter A. The waves travel through soft tissue at a known constant velocity. The distance from transducer A along centerline 63 to vessel 3 may be calculated from the travel time between the transmission of wave 62 and reception of wave 64.

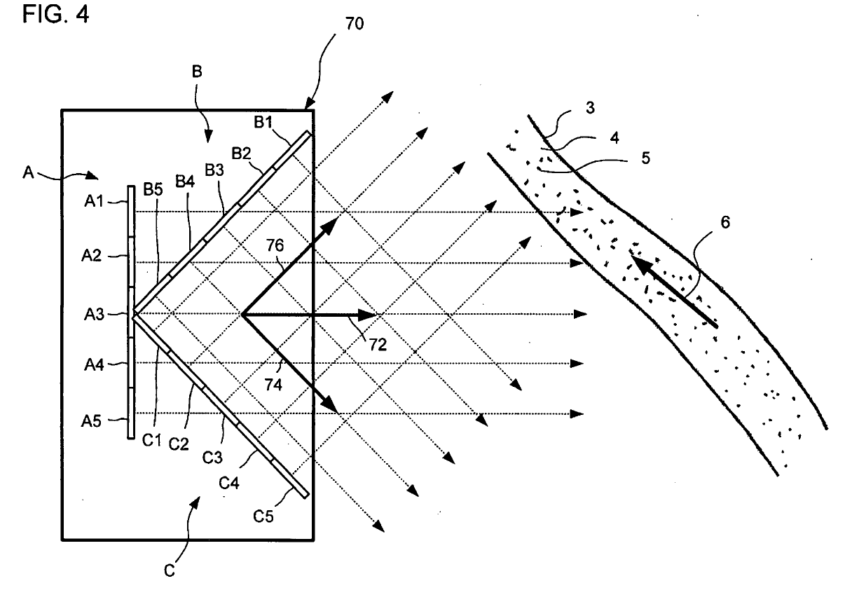

FIG. 4 illustrates Doppler sensor 70 including linear array transducers A, B and C. Doppler sensor 70 may be coupled or integrated with other components of sensing device 1. Each of transducers A, B and C is operably connected with a driver device (not shown) that powers each transducer causing each transducer to transmit an ultrasonic wave capable of travelling a certain distance to the fluid of interest and, upon reaching the fluid, reflecting a phase-shifted wave. Each of transducers A, B and C may be driven at a different frequency to distinguish the source of the reflected waves received by Doppler sensor 70. For convenience, each transducer in a linear array is referred to herein as a transducer segment. In the embodiment shown, each linear array transducer comprises five transducer segments. Transducer segments may be operably connected to be activated separately or concurrently. Separate activation of one or more transducer segments is desirable to limit power consumption. More than one transducer segment may be activated concurrently to broaden the reach of the transmitted wave. Of course, if all segments in a linear array are activated, the linear array operates as a single transducer. Doppler sensor 70 may comprise three such transducers.

Transducers A, B and C are disposed at an angle relative to each other. In one embodiment show in FIG. 4, transducers B and C are disposed at a 45 degree angle relative to transducer A and at 90 degrees relative to each other. Transducers may be positioned at different angles relative to other transducers. The positions, and angles, are selected to orient acoustic energy in directions which optimally reflect acoustic energy from a vessel. Selection is based, at least in part, on the anatomy of the patient. The anatomy of the patient may determine where to position sensing device 1, e.g., externally or implanted, positioned in front or back, and the position of sensing device 1 will determine the distance from a Doppler sensor to the vessel of interest. In one embodiment, transducers B and C are disposed at a 30 degree angle relative to transducer A and at 120 degrees relative to each other.

Transducer A includes segments A1-A5, transducer B includes segments B1-B5, and transducer C includes segments C1-C5. Each segment may transmit and receive ultrasonic energy in the form of waves. The arrows originating at each segment and projecting perpendicularly to the segment represent the direction of waves transmitted by each segment. Further, arrows 72, 74, and 76 represent the directions of waves produced by transducers A, B and C, in aggregate, respectively. The frequency of the acoustic energy is selected as a function of the distance between the transducer and the target fluid. Transducers may be energized, generally, at frequencies ranging between 2-10 MHz to reach blood conveying vessels at distances ranging, generally, between 3-20 cm, after passing through the soft tissues of the patient. In one embodiment, each of transducers A, B and C is energized at a frequency ranging between 2-10 MHz. In another embodiment, one or more segments of transducer A are energized at a frequency of 5 MHz, one or more segments of transducer B are energized at a frequency of 4.5 MHz, and one or more segments of transducer C are energized at a frequency of 5.5 MHz. A reflected wave may be measured at each segment of a linear array transducer. Each segment may be energized sequentially and may be energized a plurality of times. In other embodiments, more or less than five segments may be used to form a transducer unit. In one embodiment, between ten and fifteen segments are used.

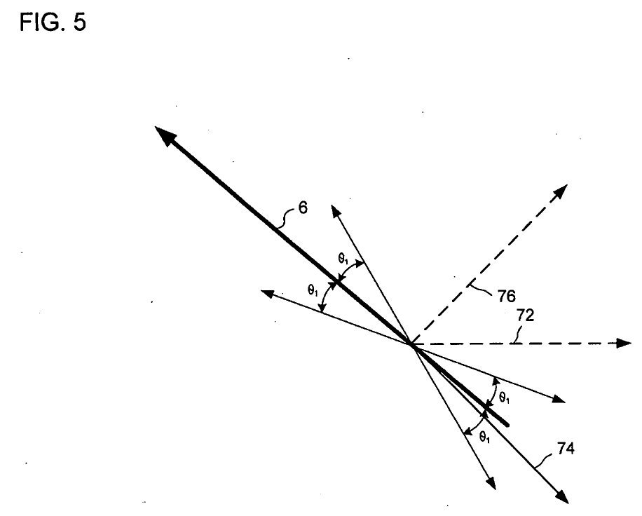

The Doppler shift, or frequency shift, is proportional to the component of the velocity vector parallel to the impinging wave. Since the Doppler shift depends from the cosine of the angle θ between the wave and the velocity vector, and the cosine function ranges between 0 and 1, signals produced by waves oriented parallel to the velocity vector produce optimal signals. In one embodiment, computing device 20 produces signals only from waves where the angle θ=θ₁ is less than or equal to 20 degrees. FIG. 5 shows conceptually the relationship between velocity vector 6 and waves having directions 72, 74 and 76 presented previously in FIG. 4. FIG. 5 also shows four arrows disposed at angle θ₁ relative to velocity vector 6. Arrow 74 is shown forming an angle with respect to velocity vector 6 which is smaller than θ₁. Consequently, waves oriented in the direction represented by arrow 74, in this case waves produced by linear array transducer B may generate usable signals. In contrast, waves oriented in the directions represented by arrows 72 and 76, corresponding to transducers A and C, will not produce usable signals.

In one embodiment, sensing device 1 includes an optical sensor assembly configured to detect the position and diameter of a vessel. Sensing device 1 may determine, based upon the position of the vessel, which transducers will not produce usable signals and, to save energy, will only transmit ultrasonic waves from transducers that may produce usable signals.

To increase the range of the Doppler sensor, additional transducers may be provided disposed different angles so that one or more of the transducers may be positioned at angles which produce waves oriented at angles that are less than or equal to 20 degrees relative to the velocity vector. In one embodiment, three transducers are arranged in the shape of a K to enable Doppler sensor 70 to obtain a sufficient number of signals even when the relative position of Doppler sensor 70 and vessel 3 change slightly with time or other factors such as a patient's activity level and posture. Reflected waves produced by one transducer may be received by more than one transducer transducer. However, since waves have frequencies corresponding to each transmitting transducer, Doppler sensor 70 is able to selectively filter signals based on the relative position of the corresponding transmitting transducer and its transmission frequency so that the Doppler shifts may be properly identified. The frequency shift corresponds to velocity as well as to direction of flow.

In one embodiment, signals from waves received by segments of linear array transducers A, B and C are filtered out when waves impinge on vessels other than the vessel of interest. The location of vessels other than the vessel of interest may be obtained in the same manner as relative position data is obtained which will be explained below. In another embodiment, computing device 20 first determines the angle θ for each segment and selectively energizes segments of transducers A, B and C only when the angle θ of a segment may generate usable signals, thereby saving energy. Furthermore, if all segments of a transducer may produce a usable signal, computing device 20 may limit the number of signals produced to save energy. For example, if all five segments are positioned to produce a usable signal, computing device 20 may select three signals to conserve 40% of the energy necessary to generate five signals.

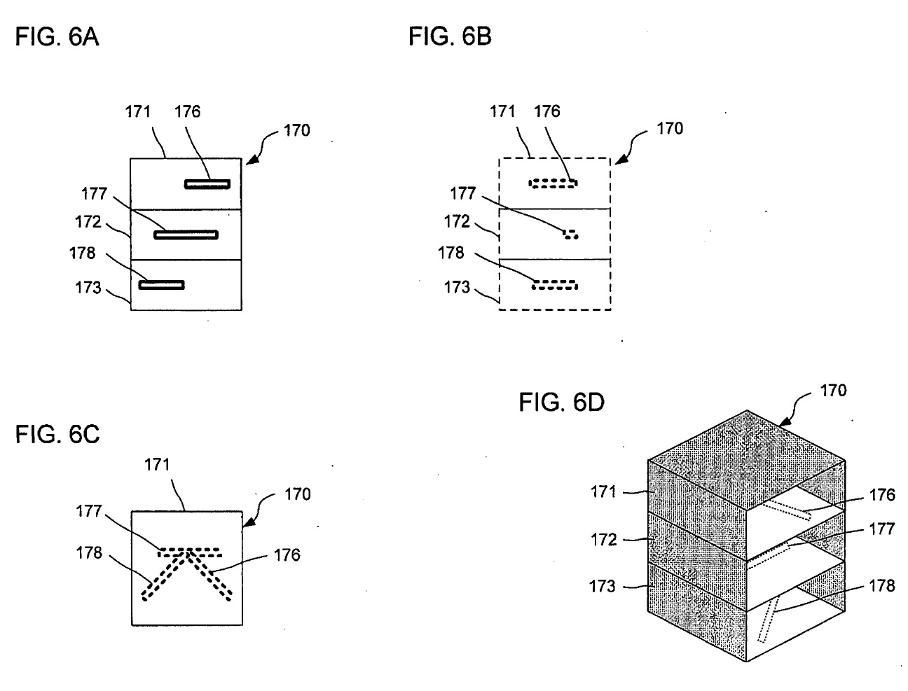

When multiple transducers comprising coils are positioned in close proximity, each transducer may interfere with the operation of the other transducers. Interference may be neutralized by an appropriate filter algorithm. However, filtering in this manner requires additional memory and energy to process the algorithm. FIGS. 6A-6D illustrate Doppler sensor 170 configured to minimize interference between transducers. Doppler sensor 170 includes transducers 171, 172 and 173 having coils 176, 177 and 178, respectively. FIGS. 6A, 6B, 6C and 6D are front, side, top, and perspective views, respectively, of Doppler sensor 171. Transducers 171, 172 and 173 enclose coils 176, 177 and 178 on three sides designated by symbol X with material configured to block electromagnetic waves, and on a fourth side designated by symbol Y with material configured to allow electromagnetic waves to pass through. Side Y is referred to herein as an electromagnetic window. Blocking material may be any suitable material including a metal, and non-blocking material may be any suitable material such as plastic. The blocking material physically eliminates interference between coils 176, 177 and 178 thereby saving energy and enabling further miniaturization of sensing device 1 by reducing memory requirements. Transducers 171, 172 and 173 are stacked rather than being laid on a common plane. Computational requirements to compensate for stacking, e.g., introducing a third-dimension to the geometric distance calculations, consume negligible resources. In many cases, stacking effects may be ignored altogether due to their negligible effect.

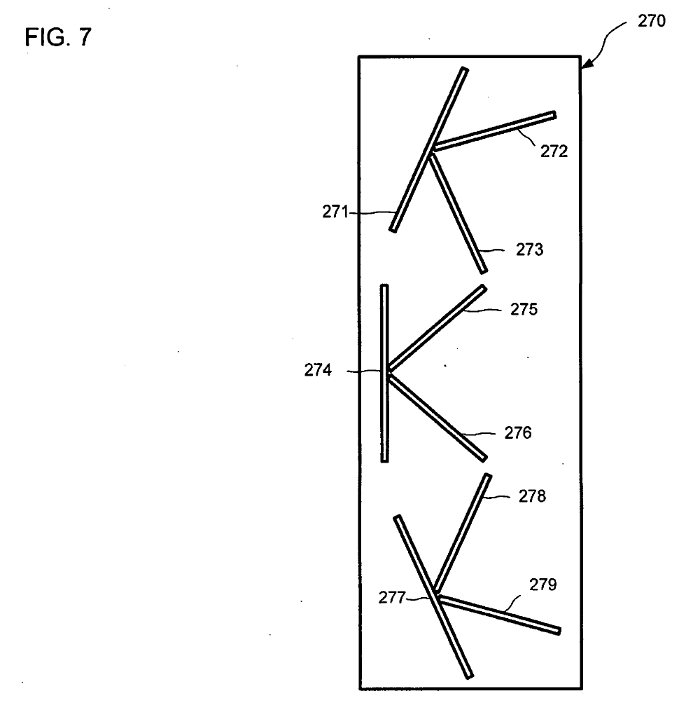

FIG. 7 illustrates a Doppler sensor according to yet another exemplary embodiment. Doppler sensor 270 includes transducers 271-279 which may be single or linear array transducers. Transducers 271-279 are positioned in the shape of three K's to provide a broader sensing reach without increasing the profile of sensing device 1 and, thus, without introducing stacking variables to the calculations. More or fewer transducers may be used to suit the shape of the housing and the location where sensing device 1 is placed. In the embodiment shown, transducers 271, 274, and 277 comprise the base of the three K-shaped arrays. Transducers 271 and 277 are disposed at 30 degree angles with respect to transducer 274, and each is disposed at 45 degree angles with respect to the remaining two legs of each K-shape array.

As discussed previously, the calculation of blood velocity requires knowledge of the incident angle θ between waves and vessel 3. Incident angle and other data characterizing the relative position of vessel 3 and a Doppler sensor may be obtained in various ways. Once obtained, it may be stored in memory as reference values. In one embodiment, the relative position data may be provided to computing device 20 through communication device 30 by an external device. The external device may transmit wirelessly communication signals to communication device 30 containing relative position data. In another embodiment, the relative position data may be provided to computing device 20 through communication device 30 by another implanted device. Other implanted devices include, without limitation, a pacemaker, a Cardiac Resynchronization Therapy (CRT) device, an implantable cardioverter defibrillator (ICD), etc. In yet another embodiment, the relative position data may be provided to computing device 20 by another sensor or sensor assembly included in sensing device 1. A sensor assembly for detecting the relative position of a vessel is provided in the above-referenced Optical Sensor Application. Once the selected signals have been determined, computing device 20 computes a blood velocity value by comparing the frequency of transmitted and received waves according to well known frequency-shift and angle algorithms or tables.

In another embodiment of a sensing device 1, a Doppler sensor and other features of the sensing device 1 are integrated with an implanted cardiac device such as a pacemaker, a Cardiac Resynchronization Therapy (CRT) device, an implantable cardioverter defibrillator (ICD), etc.

While sensing device 1 may be programmed to perform a measurement of blood velocity relatively infrequently to conserve power (e.g., once or twice per day), it should be understood that as battery technology improves, power conservation will be less of an issue, and measurements may be made more frequently. Moreover, when sensing device 1 is not implanted (i.e., is worn by the patient externally), power may be provided to sensing device 1 through connector 85, thereby eliminating the need to conserve power and permitting frequent or even continuous measurements.

2. Computing Device

Computing device 20 comprises a plurality of components. While components are described herein as if they were independent components, the components may be combined in a single device such as an application specific integrated circuit. Computing device 20 includes a processor, a memory, one or more programs, inputs and outputs. The memory may include, but is not limited to, RAM, ROM, EEPROM, flash memory or other memory technology. The processor and memory may be constructed in an integrated circuit. The integrated circuit may include one or more of Doppler sensor 60, 70, 170 and 270, and communication device 30. Further, computing device 20 may include A/D and/or D/A converters on an integrated circuit. Alternatively, A/D and/or D/A converters may be provided separately.

A program represents computer instructions directing the processor to perform tasks responsive to data. Program reside in memory. Data, including reference data and measurement data, also resides in memory. Reference data may be stored in ROM or it may be stored in RAM so that it may be modified over time, either in response to external inputs or in response to characteristics of measurement data collected over time. Protocols for responding to measurement values may also be provided. Protocols may be stored in permanent memory or may be stored in non-permanent memory such as RAM.

Computing device 20 controls Doppler sensor 60, 70, 170, or 270 and communication device 30 through inputs and outputs. Computing device 20 may control the number, frequency, power level and transmission of waves by Doppler sensor 60, 70, 170, or 270 to obtain the desired measurements using the least amount of energy.

FIG. 8 discloses system 300 for exchanging information with sensing device 1. System 300 includes sensing device 1 having, optionally, connector 85 (shown in FIG. 1A). System 300 may also include computer 302, docking station 304 operably coupled to computer 302 via cable 303 and telephone 306. In one embodiment, system 300 transmits and receives communication signals wirelessly to/from sensing device 1 based on processing performed by computing device 20.

Connector 85 is adapted to plug into docking station 304. Sensing device 1 is shown docked on docking station 304. While docked, sensing device 1 may charge energy storage device 40. Docking station 304 is operably coupled to computer 302 to update the programs and reference values stored in the memory of computing device 20 prior to placing sensing device 1 on, or in, the patient. In another embodiment, sensing device 2 is positioned externally to the patient and connector 85 is operationally coupled to an energy source to power sensing device 2 and prevent depletion of energy storage device 40.

In a further embodiment, additional sensors and devices may be coupled to sensing device 1 through connector 85. Other sensors and devices may include, without limitation, additional sensor assemblies 2, temperature sensors, pressure sensors, and accelerometers. The other devices may or may not include a computing device. Other devices may also be incorporated with sensing device 1 within housing 90. An integrated sensing device is disclosed in the above-referenced related Integrated Heart Application. The operation of sensing device 1 may be adapted to operate the additional sensors and devices by downloading into the memory of computing device 20 modified programs adapted to operate them. Downloading may occur while computing device 20 is docked in the docking station. Alternatively, new programs may be downloaded wirelessly through computing device 40.

FIG. 9 is a flowchart illustrating a program activated in computing device 20 for measuring blood parameters and performing a function responsive to measured values. At step 400, computing device 20 obtains transducer signals representative of fluid velocity from a Doppler sensor. In one embodiment, transducer signals include voltage and frequency. It should be understood that velocity signals result from waves produced by a reflecting object. In the case of blood velocity, the objects are red blood cells. It is generally understood that the velocity of red blood cells in blood accurately represent blood velocity.

Step 400 may be initiated based on cardiac cycle data to define blood velocity at a particular point in the cardiac cycle. Step 400 may also be initiated in response to an external command received through communication device 30 or as a result of detection of an abnormal condition by sensing device 1. Each of transducers A, B and C are energized sequentially. In one embodiment, transducer A transmits a wave and then switches to receive mode. Doppler sensor 70 detects the reflected waves in a manner determined by the configuration of transducer A. Transducers B and C are activated in the same manner, in sequence. In another embodiment, each transducer comprises a transmitting element and a receiving element and the transducer may, thus, be activated to simultaneously transmit and receive acoustic energy. The labelling of transducers or the energizing order are unimportant. More or fewer transducers may be utilized. The number and orientation of transducers are chosen to obtain data at angles relative to vessel 3 which produce sufficient data for the intended purpose.

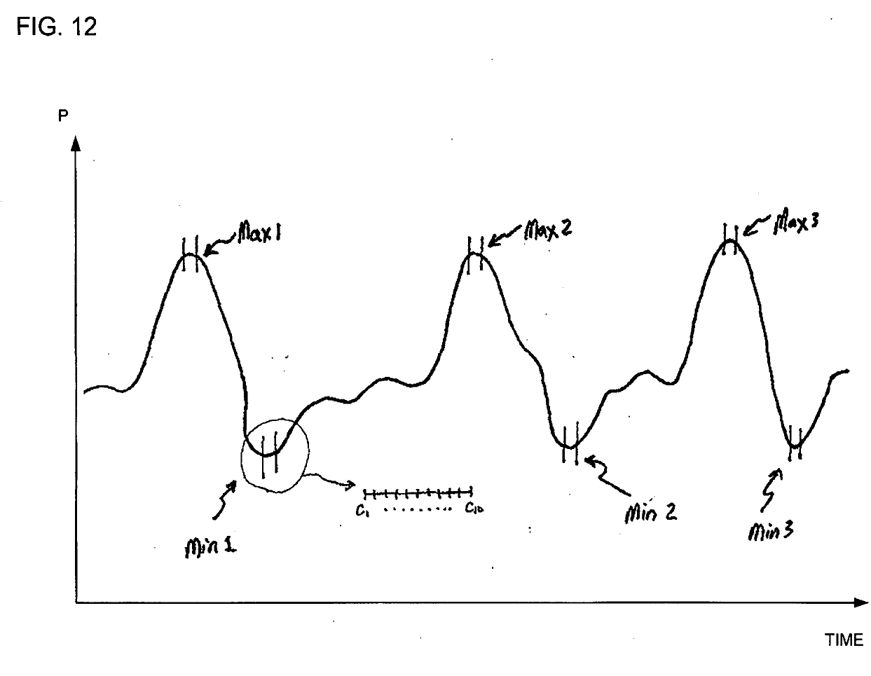

At step 402, computing device 20 processes the signals to obtain measurement values. Processing may involve removing inherent signal noise, converting signals from analog to digital form, scaling, filtering out non-selected waves, and otherwise conditioning the detected signals to convert them to measurement values. In one embodiment, measurements obtained during one cardiac cycle are averaged to obtain an average blood velocity. In another embodiment, the high and low value measurements obtained during one cardiac cycle are averaged to obtain an average blood velocity. An ECG may be used to estimate when blood flows at a maximum or minimum velocity. After processing, measured values may be stored in memory or may be analyzed to first determine whether the values should be retained. Steps 400 and 402 may be repeated as necessary to obtain sufficient measurement values to compute the desired parameters in accordance with the teachings provided herein. An embodiment of a method to calculate blood pressure based on velocity measurements is described in full detail below with reference to FIG. 12.

To save energy, it is desirable to operate Doppler sensor 70 only when it is reasonably certain that a suitable signal will be obtained. In one embodiment, low-power consumption sensors may be used to ascertain the angle of the vessel of interest relative to each transducer before Doppler sensor 70 is activated. In one embodiment, sensing device 1 includes an infrared sensor assembly 2, described with particularity in the above-referenced Optical Sensor Application. Sensor assembly 2 ascertains that sensing device 1 is positioned such that waves transmitted from transducers of the Doppler sensor intersect the velocity vector of blood at an angle approximately equal, or less than, 20 degrees. Transducers which are not positioned properly are not energized.

At step 404, computing device 20 analyzes the measurement values. Analysis may include calculation of parameter data and/or diagnosis based on measurement values. Parameter data refers to computed values such as fluid velocity, cardiac output, cardiac rhythm, etc. Diagnosis refers to the comparison of parameter values to reference values to detect an abnormal condition in the patient. Reference values are normal or expected values for the measured parameters for a particular patient. If an abnormal condition is detected, computing device 20 may communicate an alert rather than communicating measurement values as they are collected (consuming unnecessary power) or waiting to transmit measurement values until the memory is full or a predetermined transmission time is reached (exposing the patient to unnecessary danger during the waiting period).

Steps 400, 402 and 404 may be performed concurrently. The apparatus and methods of calculating velocity described above are useful in calculating the velocity of blood and other fluids. The velocity calculations in the case of continuous fluid flow do not require further calculations. However, if fluid flow is cyclical rather than continuous, additional measurements and calculations are desirable to more completely characterize flow and to diagnose abnormal conditions based on flow characteristics.

Reference values may include target values and acceptable variation ranges or limits. Reference values may also include values of measurements obtained from other sensors or from other devices through communication device 30 including, without limitation, relative position values.

Parameter values may indicate an abnormality when they fall outside reference target values or ranges. In some embodiments, parameter values may produce a statistic such as, for example, a moving average, and an abnormality would be detected when the parametric statistic differs from a reference statistic by more than an expected amount. If parameter data differs from expected values by more than a predetermined amount, computing device 20 may initiate a new measurement cycle to verify the parametric data before diagnosing an abnormality.

One abnormal medical condition is cardiac arrhythmia. Computing device 20 may be configured to perform an analysis of the measurement values to determine, for example, whether the cardiac rhythm is irregular indicating arrhythmia.

Additional abnormal medical conditions may be detected using values obtained externally or from additional sensors. Additional sensors which may be included in sensing device 1 are disclosed in the above-referenced related Optical Sensor Application, Integrated Heart Application and Health Condition Application.

At step 406, computing device 20 transmits an alert if an abnormal condition is detected, particularly a condition determined to be a serious or dangerous condition according to a prescribed protocol. The alert may be used to actuate an alarm or to alert the patient to take remedial action. A remedial action may be terminating or reducing physical activity. The alert may also provide global positioning (GPS) information to an emergency service. Referring to FIG. 6, the abnormal condition, when found to be present, may also be displayed on a computer 36 and/or transmitted via communication device 30 to a caregiver. The alert may comprise a text message or a code corresponding to the condition. Computing device 20 may also initiate a new measurement cycle and measure on a continuous basis in response to the detection of an abnormal condition.

At step 408, computing device 20 may initiate a treatment. Sensing device 1 may receive, through communication device 30, an external command to perform a treatment in response to the alert. Optionally, based on the protocol, an abnormal condition may also be used to direct a device adapted to provide treatment to deliver such treatment. Treatment may include, for example, an electric shock or a drug delivery.

At step 410, the parameter values or other information are communicated to an external device. Step 410 may be performed concurrently with any of the above steps. The parameter values may be stored in memory and transmitted wirelessly by communication device 30. The communication signal from communication device 30 may be activated on a periodic basis, in response to an abnormal condition, in response to an externally received command, whenever memory usage exceeds a predetermined amount, or whenever the energy storage level is determined to be low, the latter two conditions established to prevent data loss as a result of memory overflow or energy loss. It should also be understood that sensing device 1 may include communication devices in addition to communication device 30. For example, where communication device 30 is a cellular modem, sensing device 1 may also include a backup Bluetooth or RF communication device. Such a backup device may be desirable in situations where, after a number of attempts, it becomes apparent that the cellular modem is unable to transmit information (e.g., due to low available power, poor network coverage, etc.). In such a situation, computing device 20 may activate the backup communication device to transmit information or an alert to an alternate external receiving device.

Step 410 may be performed, for example, once an abnormal condition has been detected so as to update a caregiver on a substantially real-time basis. Step 410 may also be performed at regular intervals, such as once a day, once a week, once a month, etc. Alternatively or in addition to these transmissions, computing device 20 may be programmed to respond to requests for data received by communication device 30 (e.g., from a health care provider) by causing communication device 30 to transmit the requested data or information representing the requested data.

The communication signal may be received by equipment near the patient to alert the patient to the condition, or received remotely (such as over a network) by a healthcare provider, relative, or other predetermined recipient.

Blood velocity at a point in time depends on where that point in time is relative to the cardiac cycle of the patient. The cardiac cycle has an electrical component and a flow component. The electrical component refers to the electrical waves that cause the heart muscle to pump. The waves pass through the body and can be measured with a probe comprising electrodes that contact the body. An ECG is a good way to measure cardiac rhythms, particularly abnormal rhythms. An ECG is not, however, a reliable means for measuring the pumping ability of the heart.

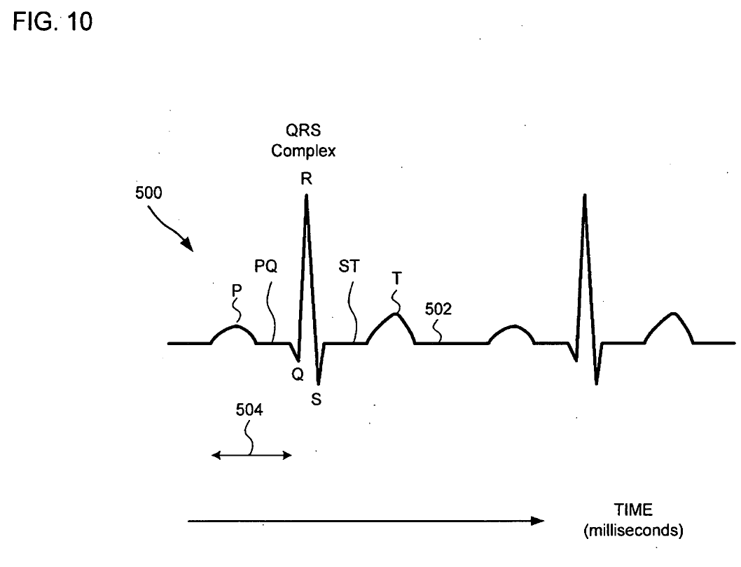

FIG. 10 illustrates an ECG graph 500 of electrical activity of the heart showing two cardiac cycles. A typical ECG consists of a P wave, a QRS complex and a T wave. An isoelectric line 502 separates a T wave and the following P wave. A PR interval 504 is measured from the beginning of the P wave to the beginning of the QRS complex. It is usually between 120 and 200 msec long. A QRS complex is about 60 to 100 msec long. The ST segment connects the QRS complex and the T wave. A typical ST segment lasts about 80 msec. In one embodiment, sensing device 1 includes an ECG sensor and an algorithm for detecting the T wave, the QRS complex and the P wave.

Cardiac cycle may be obtained in various ways. In one embodiment, cardiac cycle may be provided to computing device 20 through communication device 30 by an external device. The external device may transmit wirelessly communication signals to communication device 30 containing cardiac cycle data. In another embodiment, the cardiac cycle data may be provided to computing device 20 through communication device 30 by another implanted device. Other implanted devices include, without limitation, a pacemaker, a Cardiac Resynchronization Therapy (CRT) device, an implantable cardioverter defibrillator (ICD), etc.

In one embodiment, cardiac cycle data may be provided to computing device 20 by another sensor or sensor assembly included in sensing device 1. A sensor assembly for detecting the cardiac cycle is provided in the above-referenced Optical Sensor Application. In a further embodiment, cardiac cycle data may be provided to computing device 20 by an ECG sensor. A sensor assembly including an ECG sensor is provided in the above-referenced related Integrated Heart Application.

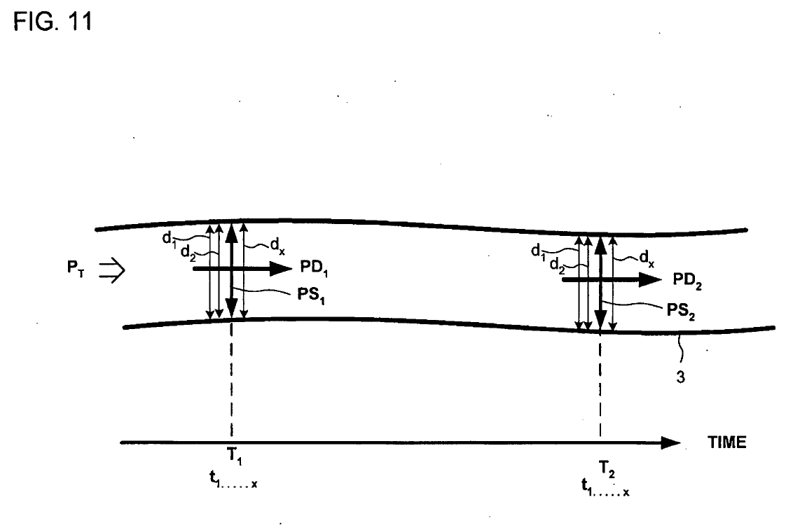

Blood flow and an embodiment of a method to characterize blood flow to calculate blood pressure will now be described with reference to FIGS. 11-12. As stated previously, blood velocity and flow vary as a function of the cardiac cycle. Velocity measurements taken in short succession may be used to characterize systolic and diastolic blood pressure. The systolic arterial pressure is the peak pressure in the arteries, which occurs near the beginning of the cardiac cycle. The diastolic arterial pressure is the lowest pressure (at the resting phase of the cardiac cycle). The time of the systolic and diastolic pressures may be estimated to predict maximum and minimum blood velocity.

In one embodiment of a method to calculate blood pressure, sensing device 1 obtains a plurality of velocity measurements at a time estimated to correspond to systolic pressure and an additional plurality of velocity measurements at a time estimated to correspond to diastolic pressure. Computing device 20 converts the velocity measurements into pressure measurements using the calculated internal surface area of aorta 3 for each measurement (determined from the diameter measurements facilitated by optical sensor assembly 2, for example) and the elapsed time between measurements, and applying the simplified Bernoulli equation for incompressible fluids: PT=PS+PD, where PT is the total pressure, PS is the static pressure, and PD is the dynamic pressure of a point in the flow stream.

Referring now to FIG. 11, at a time=T1 the dynamic pressure PD1 and the diameter d1 corresponds to the pressure determined from the velocity measurements taken under maximum blood flow conditions. At time=T2, PD2 corresponds to the pressure determined from the velocity measurements taken under minimum blood flow conditions and d2 is the diameter at time T2. In the case of aorta 3, the static pressure (depicted as force arrows directed outwardly against the outer wall of the aorta 3) under maximum flow conditions (PS1) directly corresponds to the systolic blood pressure measurement and the static pressure under minimum flow conditions (PS2) directly corresponds to the diastolic blood pressure measurement. These calculations assume laminar flow and a uniform velocity profile across the vessel. Flow velocity samples may be obtained with signals obtained from waves directed at the center of the vessel and may be used, under these assumptions, to calculate mean velocity as the velocity time integral divided of the Doppler curve divided by the flow period.

The systolic and diastolic blood pressure measurements may be derived by further computing the total pressure (PT) of blood flowing through aorta 3. PT changes as a function of time because the total pressure created by heart activity varies over time. For example, when blood is being pumped into a vessel, the total pressure created is high relative to the pressure present when the valve to the vessel is closed. In one embodiment, total pressure is derived by computing the change in pressure from the minimum flow conditions to the maximum flow conditions on the time axis. As described herein, these pressure derivations take advantage of contemporaneous diameter (and area) measurements of the vessel. This change or acceleration, in conjunction with the stroke volume and the known elasticity of aorta 3, permits computing device 20 to determine total pressure according to well known principles in the art. Thus, at time T1, the equation PT1=PS1+PD1 may be solved for PS1 and at time T2, the equation PT2=PS2+PD2 may be solved for PS2. As indicated above, PS1 and PS2 are the systolic and diastolic blood pressure measurements, respectively.

One complication for performing an accurate determination of blood pressure is the variability of the diameter of the vessel being measured. As blood is pumped through a vessel, the flexible walls of the vessel expand and contract, thereby effecting the blood pressure measurement. The effect is a result of the change in resistance force against the flow of blood which occurs as a function of the change in the vessel diameter. One embodiment of the present disclosure takes this variability into account using the technology described herein and the following methods.

As described above, blood pressure is related directly to the static pressure on the internal walls of the vessel under consideration. As is also set forth above, blood pressure (PS) is calculated using the total pressure in the vessel (PT), which is the sum of this static pressure (PS) and the dynamic pressure (PD) of the flow of blood (i.e., PT=PS+PD). The dynamic pressure is measured directly using Doppler sensor 70 as described herein. More specifically, PD is derived from blood flow (velocity) measurements using standard relationships between flow and pressure.

Static pressure is dependent, in part, on the diameter of the vessel (changes in diameter result in changes in resistance, which affect the measured static pressure). Vessel diameter is measured in this context using optical sensor assembly 2 as described herein. The area of the vessel, which is substantially circular in cross-section, is directly computed from the measured diameter. Sensing device 1 of the present disclosure computes the area of the vessel at closely spaced increments near the minimum (Min 1, Min 2, Min 3) and maximum (Max 1, Max 2, Max 3) amplitudes of the cardiac cycle of the patient. More specifically, as depicted in FIG. 12, a time velocity integral is created by computing samples of measured volume at a rate of 50 samples per second. At each sample, the present method determines the area change of vessel 3 (by measuring the diameter change using optical sensor assembly 2) and the increase or decrease in the velocity of the blood 4 flowing through vessel 3. The individual area and velocity calculations at these closely spaced samples (ten samples C1-C10 are shown in the embedded view of the samples taken at Min 1) permit individual determinations of the flow of the blood according to the relationship flow=area*velocity. It should be understood that a similar set of ten samples C1-C10 are taken at each of the peaks and valleys of the sample shown in FIG. 12. For simplicity, only one set of ten samples is depicted with an expanded time axis.

The diastolic and systolic blood pressure measurements correspond to the time velocity integral measurements at the peaks (Max 1, Max 2, Max 3) and the valleys (Min 1, Min 2, Min 3), respectively, of the samples shown in the graph of FIG. 12. In one embodiment of the present disclosure, ten samples are taken at the peaks (spaced by a few milliseconds), and ten samples are taken at the valleys (spaced by a few milliseconds). These groups of samples are taken for each of three successive pumping cycles as depicted in FIG. 12. It should be understood, of course, that more or fewer samples may be used depending upon the application. The samples are then averaged (or filtered in a manner to remove outlying samples) to determine the flow volume of each of measured sample.

The acceleration of the blood for each individual sample to the next sample in the measured sequence is determined according to the well known formula a=v²/r, where v is the velocity of blood 4 and r is the radius of vessel 3 (derived from the measurements performed by optical sensor assembly 2 as described above). The acceleration measurements are then converted into pressure according to well known principles in the art (taking into account the area change and the velocity change on the time axis). This pressure result represents the total pressure (PT), and takes into account the actual instantaneous diameter (area) of vessel 3 for each measured sample, thereby compensating for potential errors in blood pressure resulting from the flexibility of vessel 3. With PT and PD computed as described above for each sample, PS is determined by the relationship PS=PT−PD for each sample. The resulting blood pressure measurement is in units of grams per square millimeter, and may be converted to Torr units according to standard conversions (e.g., 1 Torr=1.3595e−5 Kg/mm̂2). The final PS is arrived at by averaging the ten samples at the peaks, and averaging the ten samples at the valleys. This yields three peak values and three valley values (i.e., one for each of the three cardiac cycles sampled). For each cycle, the deceleration from the peak to the valley is determined (over time), and the acceleration from a valley to the subsequent peak is determined (over time). This yields three acceleration values and three deceleration values. Each set of three is averaged to yield a final PT for acceleration and a final PT for deceleration.

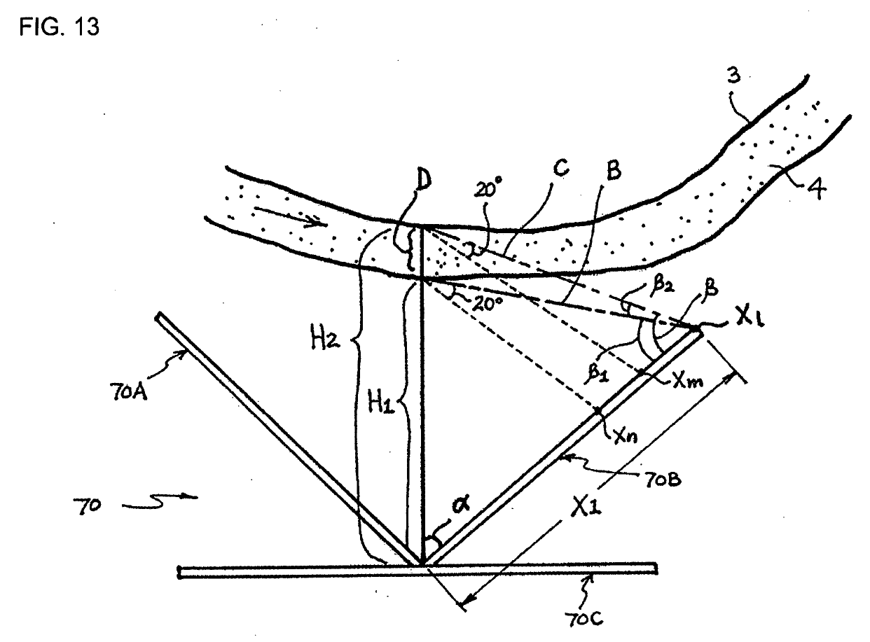

Another aspect of the present disclosure is the manner in which the Doppler measurements used in the above-described calculations are obtained. More specifically, using the measured geometry of the present sensor 1 and vessel 3 being sampled, the present system can be configured to reject irrelevant portions of the reflected waves measured by Doppler sensor 70. As described above, the waves emitted by the linear array transducers of Doppler sensor 70 travel in all directions, and reflect off of many different structures in the path of travel. Only the portion of the signal being reflected by the blood flow being measured should be used to determine velocity. As described below with reference to FIG. 13, sensor 1 can isolate the transducer segment(s) of the linear transducer array that receives this useful data.

Referring now to FIG. 13, the distances H1 and H2 are known based on the optical measurements performed by optical sensor assembly 2 as described more fully in the above-referenced related Optical Sensor Application. More specifically, because the size of sensor 1 and the location of the center of Doppler sensor 70 is known, the distance from the center of Doppler sensor 70 to each edge of vessel 3 may be calculated using the measurements provided by optical sensor assembly 2 described herein. In this example, the portion of transducer 70B of Doppler sensor 70 that provides relevant velocity information will be determined. The length of transducer 70B is represented by the designation X1, and is known because it is an actual hardware component incorporated into sensor 1. Using standard geometric relationships, computing device 20 of sensor 1 computes the length C of the triangle H2,X1,C using the angle α, which is known based on the K-shape configuration of transducers 70A, 70B, 70C. Similarly, the angle β may be determined.

As H1 was also measured using optical sensor assembly 2 as described above, the angle β1 and the length B can also be determined, yielding all of the measurements for the triangle H1,X1,B. As described above, one limitation of Doppler technology is that the angle of measurement of the reflected signal should fall within +/−20 degrees of the direction of flow. Sensor 1 uses this known characteristic of Doppler technology to project virtual points onto transducer 70B that represent the boundaries of the segment(s) on transducer 70B that will provide meaningful information about the blood velocity. More specifically, the point Xn is obtained by drawing a line at an angle of 20 degrees below side B of triangle H1,X1,B, and computing its intersection with transducer 70B. Similarly, the point Xm is obtained by drawing a line at an angle of 20 degrees below side C of triangle H2,X1,C, and computing its intersection with transducer 70B. The segment(s) of transducer 70B between points Xn and Xm and the points Xm and Xl (which is the outer end of transducer 70B) is the area of the transducer that receives reflected waves from blood 4 that provide an accurate representation of the velocity of the blood. Accordingly, the other signals detected by other segments of transducer 70B may be disregarded when computing velocity.

It should be understood that the vessel being measured in the above-described manner is continuously in motion as a result of the pumping of the heart and/or the physical activity of the person. As such, the determination of the relevant segment(s) of transducers 70A, 70B, 70C for purposes of determining blood velocity is performed frequently, and at a minimum each time a velocity sample is obtained in the above-described blood pressure computation. This data may be averaged to produce a more accurate velocity measurement.

Another aspect of the present disclosure is the manner in which the curvature of vessel 3 is accounted for in the blood pressure measurements. For each of the ten samples C1-C10 depicted at the peaks in FIG. 12, the diameter of vessel 3 is measured using optical sensor assembly 2, and the flow volume is measured using the relevant segment(s) of Doppler sensor 70. The elapsed time between samples is, of course, also known. Given the shape of vessel 3 as detected by optical sensor assembly 2 as described herein, computing device 20 can determine whether the relevant reflected portions of the Doppler signals are being reflected off blood flowing through a substantially straight or a curved portion of vessel 3. Where the sensed portion of vessel 3 is substantially straight, acceleration is derived by the relationship: acceleration=(Δflow)/(Δtime). Where the sensed portion of vessel 3 is curved, the acceleration formula is acceleration=v²/r, where r is the radius of vessel 3, but corrected by the formula w=(Δφ)/(Δtime), where φ is the curvature angle of vessel 3. The result of the formula w=(Δφ)/(Δtime) yields a percentage correction to the acceleration formula. For example, if w=0.3, then the corrected acceleration formula is a==(v²/r)*1.3. By performing the acceleration computation for each of the samples depicted in FIG. 12, the present device determines the changes in acceleration/deceleration and uses this determination in the manner described above for computing total pressure.

It should be understood that although the blood pressure computation described above refers to determining blood pressure in aorta 3, the same process may be carried out to determine blood pressure in the pulmonary artery, assuming the pulmonary artery is within the sensing range of monitoring device 1. As described in the Optical Sensor Application, monitoring device 1 distinguishes between the pulmonary artery and aorta 3 by measuring the oxygen saturation of both, and determining which vessel carries blood with higher oxygen saturation. That vessel must be aorta 3. In another embodiment of the invention, monitoring device 1 instead identifies the vessel with lower oxygen saturation as the vessel of interest (i.e., the pulmonary artery). The location and size of the pulmonary artery is then determined in the same manner as described with reference to aorta 3. With the geometry of the pulmonary artery defined, the pressure of the blood flowing through the pulmonary artery is measured as described above with reference to aorta 3.

3. Communication Device

Referring again to FIG. 8, a system 300 adapted for transmitting and receiving a communication signal. Communication device 30 is a two-way communication device, e.g. via the cellular telephone system and/or the GPS satellite system. Communication device 30 includes an antenna for transmitting and receiving communication signals. The communication signals travel wirelessly to and from one of a plurality of optional external communication devices.

An external communication device may be a computer 302 or any electronic device capable of wirelessly receiving a communication signal, such as telephone 306 which is exemplified as a cellular phone. Telephone 306 may also be an emergency service switchboard or a hospital or medical center switchboard. By communication signal is meant a signal that has one or more of its characteristics set or changed to encode information in the signal. By way of example, and not limitation, communication signals include acoustic, RF, infrared, other wireless media, and combinations of any of the above. An external communication device may also be a relay unit located externally of the patient's body, e.g. clipped to the patient's belt. The relay unit may include a receiver for receiving the transmissions from communication device 30, and a transmitter for re-transmitting the communication signal to another external communication device. The relay unit may also be stationary and hardwired for connection to the internet or direct connection to a healthcare provider's computer. Likewise, the relay unit may receive a communication signal from a healthcare provider and transmit the signal to communication device 30.

The communication signal from communication device 30 may include a voice message, a text message, and/or measured data. The communication received by communication device 30 may include data, such as updated reference data, or commands. A command may include, for example, instructions to computing device 20 for performing a task such as providing a treatment to the patient, collecting and transmitting additional data, or updating the reference data.

4. Energy Storage Device

Referring again to FIGS. 1A, 1B and 1C, a system for recharging energy storage device 40 may be provided. Computing device 20 receives energy from energy storage device 40. Energy storage device 40 includes an energy storage component such as a battery. Optionally, sensing device 1 may also include an energy coupler for receiving energy from an external source to charge energy storage device 40.

One example of an energy coupler is an electromagnetic device, such as induction coils 42, for receiving external electromagnetic signals 44 and converting such signals into electrical energy for recharging the energy storage component. An external electromagnetic device 46 generates electromagnetic signal 44 which is received and converted into electrical energy by energy storage device 40. Energy storage device 40 may provide a charge signal to computing device 20. Computing device 20 may compare the charge signal to a reference charge signal and initiate a low charge communication signal for alerting the patient and/or healthcare providers. Alternatively, a detector, such as a voltage sensor, may be used to monitor the charge of energy storage device 40 and provide a signal to computing device 20 when the charge falls below a threshold. Electromagnetic device 46 may be placed near sensing device 1 to charge energy storage device 40.

Energy may instead, or additionally, be provided in the form of ultrasonic vibrations. For example, a piezoelectric transducer may be included in sensing device 1. An ultrasonic vibration may be provided externally. The transducer generates electricity when driven by ultrasonic vibrations.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A sensing device configured to measure blood pressure, including:
a Doppler sensor having a plurality of transducers for emitting source waves and detecting reflected waves, the Doppler sensor having an associated reference location;
an optical sensor including a plurality of emitters and a plurality of detectors for generating a plurality of signals representing a first distance between the reference location and a near wall of the vessel and a second distance between the reference location and a far wall of the vessel; and
a computing device configured to, for each of a plurality of pressure calculations, determine the first and second distances to compute an area of the vessel and to determine a segment of a transducer that detects reflected waves from blood flowing through the vessel to thereby determine the velocity of the blood, the velocity and area being used to compute blood pressure.
